(19) United States
(12) Patent Application Publication
(10) Pub. No.: US 2023/0038866 A1
(43) Pub. Date: Feb. 9, 2023

(54) RAPID AND SPECIFIC EX-VIVO DIAGNOSIS OF CENTRAL NERVOUS SYSTEM LYMPHOMA

(71) Applicant: ARIZONA BOARD OF REGENTS ON BEHALF OF ARIZONA STATE UNIVERSITY, Scottsdale, AZ (US)

(72) Inventors: Xiaodong Qi, Chandler, AZ (US); Hao Yan, Chandler, AZ (US); Peter Nakaji, Phoenix, AZ (US); Joseph Georges, Mesa, AZ (US); Xiaowei Liu, Tempe, AZ (US)

(21) Appl. No.: 17/814,407

(22) Filed: Jul. 22, 2022

Related U.S. Application Data
(60) Provisional application No. 63/225,305, filed on Jul. 23, 2021.

(51) Int. Cl. G01N 33/53; C12N 15/115

(52) U.S. Cl. CPC G01N 33/5308; C12N 15/115; C12N 2310/16

(57) ABSTRACT

Disclosed are methods of detecting and treating suspected B-cell lymphoma.

### CROSS-REFERENCE TO RELATED APPLICATIONS

The present application claims priority to U.S. Provisional Patent Application No. 63/225,305 filed Jul. 23, 2021, the entire contents of which are hereby incorporated by reference.

### REFERENCE TO AN ELECTRONIC SEQUENCE LISTING

The contents of the electronic sequence listing (112624.01341.xml Size: 2,041 bytes; and Date of Creation: Jul. 18, 2022) is herein incorporated by reference in its entirety.

### STATEMENT REGARDING FEDERALLY SPONSORED RESEARCH

This invention was made with government support under R21 EB020237 awarded by the National Institutes of Health. The government has certain rights in the invention.

### FIELD OF THE INVENTION

The field of the invention relates to the detection of B-cell lymphoma. In particular, the field of the invention relates to the use of aptamers specific for lymphoma cells to intraoperatively confirm the presence of lymphoma.

### BACKGROUND OF THE INVENTION

Brain tumors remain a significant clinical problem with new cases affecting over 60,000 Americans each year. Diagnosis of tumor type is the critical determinant that drives treatment. In neurosurgery, brain tumors are not definitively diagnosed until a surgical biopsy is histologically examined by a pathologist. During surgery, these diagnoses are made using the clinical standard of frozen sections, the results of which can dictate the surgical strategy—including whether a tumor is excised or is better left to be treated post-operatively. For example, non-operative lesions such as B cell lymphomas are indistinguishable from operative lesions such as high-grade astrocytomas at a gross morphological level and diagnosis can even be challenging using frozen sections alone. As a result, revision of diagnosis may occur and impede patient care. When a definitive diagnosis cannot be made from examination of frozen sections alone, standard of care dictates that more specific immunohistochemistry (IHC) staining must be ordered. However, this mid-twentieth century technique requires 1-2 days to provide a diagnosis. Initial non-definitive diagnoses can lead to longer hospital admission times and additional surgeries for patients, resulting in further risks for patient morbidity and increased health care costs. Accordingly, improved tools for specific intraoperative brain tumor diagnosis are needed.

BRIEF SUMMARY OF THE INVENTION

Disclosed herein are methods and compositions for the rapid detection of B-cell lymphomas in a tumor sample of a subject in need thereof, and/or to distinguish B-cell lymphoma from other types of tumors/malignancies. The methods include contacting a tumor sample from the subject with a labeled aptamer comprising SEQ ID NO: 1, or an aptamer at least about 90% identical to SEQ ID NO: 1 to generate a treated tumor sample, washing the treated tumor sample, and imaging the treated tumor sample to detect the presence or absence of the labeled aptamer, wherein the presence of the labeled aptamer on the treated tumor sample is indicative of a B-cell lymphoma. In some embodiments, the tumor sample comprises a brain tumor sample. In some embodiments, the tumor sample comprises a biopsy slice.

Also disclosed are methods of treating a subject with a tumor comprising contacting a tumor sample from a subject with an aptamer having at least 90% sequence identity to SEQ ID NO. 1, wherein the aptamer comprises a detectable label, to generate a treated tumor sample, washing the contacted tumor sample, imaging the treated tumor sample to detect the labeled aptamer, wherein the presence of the labeled aptamer on the tumor sample is indicative of B-cell lymphoma and treating the patient based on the presence or absence of aptamer in the treated sample. In some embodiments, the tumor sample comprises a brain tumor sample. In some embodiments, the tumor sample comprises a biopsy slice.

Also disclosed herein are methods of preparing a tumor sample for imaging. In some embodiments, the methods comprise contacting the tumor sample with a labeled aptamer having at least 90% sequence identity to SEQ ID NO. 1 to create a treated tumor sample, and washing the treated tumor sample. In some embodiments, the tumor sample comprises brain tumor sample. In some embodiments, the tumor sample comprises a biopsy slice.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A-1I. ″Twenty-minute staining protocol. Images of xenograft biopsies with RFP-expressing tumor cells stained with 3.0-μM (A-C), (D-F), and 0.3-μM (G-I) TD05-488 aptamer. The 0.3-μM concentration identified significantly fewer tumor cells than the 1.0- and 3.0-μM concentrations. Fluorescent artifacts without ring-like staining were present across all aptamer samples (arrowheads). Bar=20 λm. **p<0.001.

FIG. 2A-2I. Twenty-minute staining with 3.0-μM (A-C) and 1.0-μM (D-F) concentrations did not identify significantly more lymphoma cells than 10-minute staining with 1.0-μM TD05-488 (G-I). Bar=20 μm.

FIG. 3A-3I. Ten-minute staining with 1.0-μM TD05-488 (A-C) identified a significantly greater percentage of tumor cells than the 5-minute staining with the 1.0-μM (D-F) and 3.0-04 (G-I) concentrations. Bar=20 μm. **p<0.001.

FIG. 4. Astrocytoma negative control. Ten-minute staining protocol with 1-μM TD05-488 yielded a false-positive rate of 0.81% ±1.75% from astrocytoma xenograft biopsies. Note the areas with fluorescent artifacts that lack a ring-like staining pattern (arrows). Bar=20 μm. **p<0.001.

FIG. 5A-5D. Nonspecific aptamer and normal brain negative controls. Lymphoma biopsies incubated with a nonspecific fluorescent aptamer (A and B) and normal brain incubated with 1.0-μM TD05-488 (C and D) yielded a false-positive rate of 2.71% ±3.72% and 0.80% ±1.27%, respectively, compared to 76.7% ±15.1% of lymphoma cells labeled by TD05-488. Bar=20 μm.

FIG. 6. An image series of 27 randomly selected images that was distributed to pathologists and neurosurgeons for interpretation. Lymphoma samples labeled with asterisks in upper left corner. Reviewers did not receive labeled images. Scale bars equal 20 μm.

FIG. 7A-7H. Standard Histologic Staining of Xenograft Biopsies Compared to TD05-488 Aptamer. Hematoxylin and Eosin staining show regions of hypercellular tumor (A&E). Lymphoma sections show strong CD20 staining (B&C), in contrast to glioma without visualized CD20 staining (F&G). High magnification imaging shows ring-like staining pattern of CD20 (C), and similar aptamer staining pattern of red-fluorescent protein-expressing B-cell lymphoma cells (D). High magnification image of glioma specimen lacks CD20 staining (G), and lack of ring-like aptamer staining in red fluorescent protein-expressing glioma specimen (H). Scale bars equal 20 μm.

### DETAILED DESCRIPTION OF THE INVENTION

The present invention is described herein using several definitions, as set forth below and throughout the application.

Definitions

The disclosed subject matter may be further described using definitions and terminology as follows. The definitions and terminology used herein are for the purpose of describing particular embodiments only and are not intended to be limiting.

As used herein, the term aptamer refers to a class of nanomolecules engineered to bind targets molecules. Aptamers may be constructed from naturally occurring or non-naturally occurring nucleic acids and/or amino acids. In some embodiments, aptamers comprise one or more detectable labels.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or 13 or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of treatment for a disease or disorder associated with a suspected tumor, such as a central nervous system tumor. A subject in need thereof may include a subject having a cancer that is characterized by gross abnormality visible by X-ray, computerized tomography (CT), or magnetic resonance imaging (MRI), but which has not been diagnosed as a central nervous system (CNS) tumor by histology or immunofluorescence. In some embodiments, the tumor comprises a lymphoma, and in some embodiments, the lymphoma comprises a B-cell lymphoma. In some embodiments, the tumor comprises a CNS lymphoma in the brain of the subject.

The term “subject” may be used interchangeably with the terms “individual” and “patient” and includes human and non-human mammalian subjects.

The disclosed treatment methods may be utilized to treat diseases and disorders associated with suspected central nervous system (CNS) cancer which may include, but are not limited to cell proliferative diseases and diseases and disorders such as brain cancers. Suitable cancers for treatment by the disclosed methods may include, but are not limited to astrocytoma, oligodendroglioma, mixed gliomas, ependymoma, medulloblastoma, pineal parenchymal tumors, meningeal tumors, germ cell tumors, or craniopharyngioma. In some embodiments, the cancer comprises a lymphoma such as a B-cell lymphoma.

The disclosed methods may be used to detect the presence of lymphoma, such as B-cell lymphoma. The disclosed methods may be used to detect B-cell lymphoma in any tissue. In some embodiments, the disclosed methods are used to detect tumors located in several nervous tissues including, but not limited to: tumors located in the brain, spinal cord, or the meninges including the leptomeninges.

By way of example, but not by way of limitation, the disclosed methods may be utilized to detect the presence of CNS lymphoma in a tumor sample from a subject. In some embodiments, the tumor sample comprises a liquid sample, such as blood, cerebrospinal fluid, or lymph. In some embodiments, the tumor sample comprises a solid tumor sample such as a tissue sample, prepared, for example, by fresh sectioning without fixation or being embedded in a substrate, frozen sections, frozen sections that have been embedded in a paraffin based substrate, frozen sections that have been embedded in a non-paraffin based substrate.

By way of example, but not by way of limitation, the disclosed detection and preparation methods may be used on tumor samples (e.g., tissue samples) including tissues obtained by: surgical resection or slicing, needle biopsies, or punch biopsies.

By way of example, but not by way of limitation, the disclosed detection or preparation methods may be used on tumor samples (e.g., tissue samples) obtained from cell culture or organoid cell culture, or a similar method of artificially creating tissue from isolated cells.

As used herein the term “treated tumor sample” refers to a tumor sample or collection of cells that has been contacted with a labeled aptamer of the present disclosure. In some embodiments, the labeled aptamer binds to the tumor sample, and remains bound, even after washing, and is indicative of the presence of a tumor, such as a B-cell lymphoma. Tumor samples include, but are not limited to biopsy slices, needle aspirates, punches, cells, organoids, etc. In some embodiments, a treated tumor sample may be referred to as stained or labeled.

Suitable detectable labels for the imaging of the aptamer in the disclosed methods, are not limited to, but include: fluorescent labels, luminescent labels, radioisotopic labels, and labels that comprise an enzyme capable of conversion of a substrate leading to detection.

The disclosed methods relate to the use of an aptamer comprising SEQ ID NO:1, or an aptamer with greater than 90% sequence similarity to SEQ ID NO:1. In some embodiments, the aptamer has about 91, 92, 93, 94, 95, 96, 97, 98, 99 or 100% sequence similarity with SEQ ID NO: 1. In some embodiments, the aptamer nucleic acid sequence consists of SEQ ID NO: 1.

SEQ ID NO:1 has the following sequence: AGGAGGATAGTTCGGTGGCTGTTCAGGGTCTCCTCCT; in some embodiments, the molecule of SEQ ID NO:1 comprises a detectable label.

Rapid and Specific Diagnosis of Central Nervous System Lymphoma

In some embodiments, the present disclosure provides a method of detecting the presence of lymphoma, such as B-cell lymphoma, the method comprising: contacting tumor samples, such as brain biopsy sections, with an aptamer comprising at least 90% sequence identity to SEQ ID NO. 1, wherein the aptamer comprises a detectable label, to generate treated tumor sample, washing the treated tumor samples, and imaging the treated tumor samples.

As used herein, the term aptamer refers to a class of nanomolecules engineered to bind targets molecules. Aptamers may be constructed from naturally occurring or non-naturally occurring nucleic acids and/or amino acids. In some embodiments, aptamers comprise one or more detectable labels.

Suitable detectable labels for the imaging of the aptamer in the disclosed methods, are not limited to, but include: fluorescent labels, luminescent labels, radioisotopic labels, and enzymes.

In some embodiments the tumor is a brain tumor, a spinal cord tumor, an eye tumor, a meningeal tumor or a leptomeningeal tumor. Suitably, the tumor sample may comprise a biopsy, or biopsy section or slice of a tumor described herein. A brain biopsy is the removal of a small piece of brain tissue for diagnosis of abnormalities.

In some embodiments of the method, the aptamer has a concentration of between 0.3 mM and 3 mM, inclusive.

In some embodiments of the method, the aptamer has a concentration of between 0.3 mM and 1 mM, inclusive.

In some embodiments of the method, the aptamer has a concentration of between 1mM and 3 mM.

In some embodiments of the method, the biopsy sections are contacted with the aptamer for between 5 and 20 minutes, inclusive.

In some embodiments of the method, the biopsy sections are contacted with the aptamer for between 5 and 10 minutes, inclusive.

In some embodiments of the method, the biopsy sections are contacted with the aptamer for between 10 and 20 minutes, inclusive.

In some embodiments of the method, the biopsy sections are contacted with an agent to block non-specific binding (blocking agents), prior to or at the same time as contact with the aptamer. An agent which blocks non-specific binding decreases unwanted background staining or binding of the aptamer to nonspecific targets. Agents to block non-specific binding are known in the art and can include tRNA.

In some embodiments of the method, the biopsy sections are contacted with yeast transfer RNA (tRNA) in addition to the aptamer.

In some embodiments of the method, the biopsy sections are contacted with yeast transfer RNA (tRNA) with a concentration of 0.1 mg/ml in addition to the aptamer.

In some embodiments, this disclosure provides a method of treating lymphoma in a subject in need thereof, the method comprising: contacting tumor samples from the subject with an aptamer comprising at least 90% sequence identity to SEQ ID NO. 1, wherein the aptamer comprises a detectable label, to create treated tumor sample, washing treated tumor sample, imaging the treated tumor sample, and treating the subject based on the presence or absence of aptamer in the tumor sample. In some embodiments, the tumor sample comprises a biopsy slice or section.

In some embodiments of the method of treatment, imaging the treated tumor sample reveals the presence of aptamer in the tumor sample, and the method comprises treating the subject with a non-surgical medical intervention.

In some embodiments of the method of treatment comprising non-surgical intervention, the non-surgical medical intervention is at least one modality selected from the group consisting of chemotherapy, radiation, bone marrow transplant, and immunotherapy.

In some embodiments of the method of treatment, imaging the stained biopsy sections reveals the absence of aptamer staining in the tumor sample, and the method comprises treating the subject with a surgical intervention.

In some embodiments of the method of treatment comprising surgical intervention, the surgical intervention is resection of the suspected tumor tissue.

In some embodiments of the method of treatment comprising surgical intervention, the surgical intervention is maximal safe resection of the suspected tumor tissue.

In some embodiments of the method of treatment comprising surgical intervention, the surgical intervention is performed as an adjunct to the surgery to collect the biopsy.

In some embodiments of the method of treatment, the aptamer has a concentration of between 0.3 mM and 3 mM, inclusive.

In some embodiments of the method of treatment, the aptamer has a concentration of between 0.3 mM and 1 mM, inclusive.

In some embodiments of the method of treatment, the aptamer has a concentration of between 1mM and 3 mM.

In some embodiments of the method of treatment, the biopsy sections are contacted with the aptamer for between 5 and 20 minutes, inclusive.

In some embodiments of the method of treatment, the biopsy sections are contacted with the aptamer for between 5 and 10 minutes, inclusive.

In some embodiments of the method of treatment, the biopsy sections are contacted with the aptamer for between 10 and 20 minutes, inclusive.

In some embodiments of the method of treatment, the biopsy sections are contacted with yeast transfer RNA (tRNA) in addition to the aptamer.

In some embodiments of the method of treatment, the biopsy sections are contacted with yeast transfer RNA (tRNA) with a concentration of 0.1 mg/ml or about 0.1 mg/ml in addition to the aptamer. Also provided herein are methods for preparing a tumor sample for imaging.

In some embodiments, the methods comprise contacting the tumor sample with an aptamer comprising at least 90% sequence identity to SEQ ID NO. 1, wherein the aptamer comprises a detectable label, to create a treated tumor sample; washing the treated tumor sample; and imaging the treated tumor sample.

In some embodiments of the method of preparing a tumor sample for imaging, the tumor comprises a brain tumor.

In some embodiments of the method of preparing a tumor sample for imaging, the tumor sample comprises a biopsy section.

In some embodiments of the method of preparing a tumor sample for imaging, the aptamer has a concentration of between 0.3 mM and 3 mM, inclusive.

In some embodiments of the method of preparing a tumor sample for imaging, the aptamer has a concentration of between 0.3 mM and 1 mM, inclusive.

In some embodiments of the method of preparing a tumor sample for imaging, the aptamer has a concentration of between 1mM and 3 mM.

In some embodiments of the method of preparing a tumor sample for imaging, the tumor sample is contacted with the aptamer for between 5 and 20 minutes, inclusive.

In some embodiments of the method of preparing a tumor sample for imaging, the tumor sample contacted with the aptamer for between 5 and 10 minutes, inclusive.

In some embodiments of the method of preparing a tumor sample for imaging, the biopsy section is contacted with the aptamer for between 10 and 20 minutes, inclusive.

In some embodiments of the method of preparing a tumor sample for imaging, the tumor sample is contacted with yeast transfer RNA (tRNA) in addition to the aptamer. In some embodiments, the tRNA has a concentration of 0.1 mg/ml.

### EXEMPLARY EMBODIMENTS

1. A method of detecting the presence of B-cell lymphoma in a tumor sample from a subject, the method comprising:

contacting the tumor sample with an aptamer comprising at least 90% sequence identity to SEQ ID NO. 1, wherein the aptamer comprises a detectable label, to create a treated tumor sample;

washing the treated tumor sample; and imaging the treated tumor sample, wherein the presence of the labeled aptamer is indicative of B-cell lymphoma.

2. The method of embodiment 1, wherein the tumor is one or more of a brain tumor, a spinal cord tumor, an eye tumor, a meningeal tumor, or a leptomeningeal tumor.

3. The method of embodiment 2, wherein the tumor comprises a brain tumor.

4. The method of any of the previous embodiments, wherein tumor sample comprises a biopsy section.

5. The method of any of the preceding embodiments, wherein the aptamer has a concentration of between 0.3 mM and 3 mM, inclusive.

6. The method of any of the preceding embodiments, wherein the aptamer has a concentration of between 0.3 mM and 1 mM, inclusive.

7. The method of any of the preceding claims, wherein the aptamer has a concentration of between 1mM and 3 mM

8. The method of any of the preceding embodiments, wherein the tumor sample is contacted with the aptamer for between 5 and 20 minutes, inclusive.

9. The method of any of the preceding embodiments, wherein the tumor sample is contacted with the aptamer for between 5 and 10 minutes, inclusive.

10. The method of any of the preceding embodiments, wherein the tumor sample is contacted with the aptamer for between 10 and 20 minutes, inclusive.

11. The method of any of the preceding embodiments, wherein the tumor sample is contacted with an agent to block non-specific binding in addition to the aptamer.

12. The method of any of the preceding embodiments, wherein the tumor sample is contacted with yeast transfer RNA (tRNA) in addition to the aptamer.

13. The method of embodiment 9, wherein the tRNA has a concentration of 0.1 mg/ml.

14. The method of any of the preceding embodiments, wherein the label is a fluorescent label, luminescent label, radioisotopic label, or a label that comprises an enzyme capable of a colorimetric conversion of a substrate.

15. The method of embodiment 1, wherein the subject has a brain tumor, the tumor sample comprises a fresh biopsy section, the aptamer is SEQ ID NO: 1 and comprises a fluorescent label, and the method, including imaging, is completed in 20 minutes or less.

16. A method of treating B-cell lymphoma in a subject in need thereof, the method comprising:

contacting a tumor sample from the subject with an aptamer with at least 90% sequence identity to SEQ ID NO. 1, wherein the aptamer comprises a detectable label, to create treated sample, washing the treated tumor sample, imaging the treated tumor sample, and treating the patient based on the presence or absence of aptamer in the treated tumor sample.

17. The method of embodiment 15, wherein imaging the imaging the treated tumor sample reveals the presence of aptamer staining, wherein treating the subject comprises a non-surgical medical intervention.

18. The method of embodiment 16, wherein the non-surgical medical intervention is at least one modality selected from chemotherapy, radiation, bone marrow transplant, and immunotherapy.

19. The method of embodiment 15, wherein imaging the stained biopsy sections reveals the absence of aptamer staining in the stained biopsy sections, and wherein treating the subject comprises a further surgical intervention.

20. The method of embodiment 18, wherein the further surgical intervention is resection of the suspected tumor tissue.

21. The method of embodiment 18 or 19, wherein the further surgical intervention is maximal safe resection of the suspected tumor tissue.

22. The method of any of embodiments 18-20, wherein the further surgical intervention begins before the conclusion of the surgery to collect tumor sample.

23. The method of any of embodiments 15-21, wherein the aptamer has a concentration of between 0.3 mM and 3 mM, inclusive.

24. The method of any of embodiments 15-22, wherein the aptamer has a concentration of between 0.3 mM and 1 mM, inclusive.

25. The method of any of embodiments 15-22, wherein the aptamer has a concentration of between 1mM and 3 mM.

26. The method of any of embodiments 15-24, wherein the biopsy sections are contacted with the aptamer for between 5 and 20 minutes, inclusive.

27. The method of any of embodiments 15-25, wherein the biopsy sections are contacted with the aptamer for between 5 and 10 minutes, inclusive.

28. The method of any of embodiments 15-25, wherein the biopsy sections are contacted with the aptamer for between 10 and 20 minutes, inclusive.

29. The method of any of embodiments 15-27, wherein the biopsy sections are contacted with yeast transfer RNA (tRNA) in addition to the aptamer.

30. The method of any of embodiments 15-28 wherein the biopsy sections are contacted with yeast transfer RNA (tRNA) with a concentration of 0.1 mg/ml in addition to the aptamer.

31. The method of any one of embodiments 15-29, wherein the tumor sample comprises a biopsy slice.

32. The method of any one of embodiments 15-30, wherein the tumor comprises a brain tumor.

33. A method of preparing a tumor sample for imaging, the method comprising:

contacting the tumor sample with an aptamer comprising at least 90% sequence identity to SEQ ID NO. 1, wherein the aptamer comprises a detectable label, to create a treated tumor sample; and washing the treated tumor sample.

34. The method of embodiment 32, wherein the tumor comprises a brain tumor.

35. The method of any of embodiments 32 or 33, wherein tumor sample comprises a biopsy section.

36. The method of any of embodiments 32-34, wherein the aptamer has a concentration of between 0.3 mM and 3 mM, inclusive.

37. The method of any of embodiments 32-35, wherein the aptamer has a concentration of between 0.3 mM and 1 mM, inclusive.

38. The method of any of embodiments 32-36, wherein the aptamer has a concentration of between 1mM and 3 mM.

39. The method of any of embodiments 32-37, wherein the tumor sample is contacted with the aptamer for between 5 and 20 minutes, inclusive.

40. The method of any of embodiments 32-38, wherein the tumor sample contacted with the aptamer for between 5 and 10 minutes, inclusive.

41. The method of any of embodiments 32-39, wherein the biopsy section is contacted with the aptamer for between 10 and 20 minutes, inclusive.

42. The method of any of embodiments 32-40, wherein the tumor sample is contacted with yeast transfer RNA (tRNA) in addition to the aptamer.

### EXAMPLES

The following Examples are illustrative and should not be interpreted to limit the scope of the claimed subject matter.

### Example 1—Rapid and Specific Diagnosis of Central Nervous System Lymphoma

Abstract

The inventors provide a novel diagnostic method to identify B-cell lymphoma cells and demonstrate the efficacy of the method by differentiating CNS Lymphoma from other intracranial malignant cells. The inventors have developed novel lymphoma—specific aptamers and demonstrate rapidly and specifically diagnosing xenografted orthotopic human CNS lymphoma at the time of biopsy (ex vivo).

Introduction

Differentiating central nervous system (CNS) lymphoma from other intracranial malignancies remains a clinical challenge in surgical neuro-oncology. Advances in clinical fluorescence imaging contrast agents and devices may mitigate this challenge. Aptamers are a class of nanomolecules engineered to bind cellular targets with antibody-like specificity in a fraction of the staining time. Here, it is determine if immediate ex vivo fluorescence imaging with a lymphoma-specific aptamer can rapidly and specifically diagnose xenografted orthotopic human CNS lymphoma at the time of biopsy.

Methods

The TD05-488 aptamer has the following sequence:

(SEQ ID NO: 1)
5′/5Alex488N/AGGAGGATAGTTCGGTGGCTGTTCAGGGTCTCCTCC
T-3′.

Lymphoma cells were implanted intracranially into athymic nude mice; xenographs were collected and placed in aptamer solutions.

The annealed aptamers were mixed with yeast transfer RNA (tRNA; 0.1 mg/ml), which was used to block nonspecific binding. One milliliter of the prepared aptamer mixture was used to submerse the tissue slices in a glass bottom dish and incubated on ice for 20, 10, or 5 minutes. Staining solution was then aspirated from the staining dish, and the tissue slices were quickly rinsed with 1 ml of ice-cold aptamer binding buffer for 1 minute before fluorescence imaging.

Results

In this embodiment, the use of 1.0 micromolar TD05-488 and staining for 11 minutes provided the most accurate diagnostic protocol.

This protocol allowed clinicians to positively identify all positive control lymphoma images without misdiagnosing negative control images from astrocytoma and normal brain.

### Example 2—Provision of Rapid and Specific Ex Vivo Diagnosis of Central Nervous System Lymphoma from Rodent Xenograft Biopsies by a Fluorescent Aptamer

Treatment for cancer patients often relies on definitive histopathological diagnosis from biopsies.¹ However, diagnostic stains are often time-consuming and can delay patient-specific treatment plans, including decisions regarding resection. Brain tumors such as astrocytomas are often debulked in surgical candidates, whereas central nervous system (CNS) lymphoma is generally not surgically debulked and is best treated with chemoradiation.² Preoperatively, CNS lymphoma can be indistinguishable from other malignant brain tumors with imaging modalities such as Mill and CT, and minimally invasive techniques such as flow cytometry can yield inconclusive results.³ Therefore, direct tissue sampling is often required to definitively diagnose CNS lymphoma.^{4.5}

Open biopsy and stereotactic needle biopsy are common techniques for sampling brain tumors.⁶ Patients with tumors amenable to surgery typically undergo concomitant open biopsy with tumor resection.^{7,8} During open biopsy, a frozen section is often obtained early in the case to guide the surgical plan. If the frozen section suggests CNS lymphoma, the surgery is often halted. If the tumor diagnosis on frozen section is other than CNS lymphoma, the surgery proceeds to maximal safe resection. However, the delay from waiting for the frozen section result can unnecessarily extend surgery time. Furthermore, frozen sections occasionally fail to differentiate CNS lymphoma from tumors that benefit more from resection such as astrocytomas.^{9,10} This denies information critical for good decision-making at open biopsy. These cases require special stains to obtain a specific diagnosis, a process that can take days to weeks.¹¹ The lack of accurate and specific histopathological information can lead to inappropriate termination of surgery or resection of a tumor best treated without surgery. Therefore, rapid and specific intraoperative diagnosis of CNS lymphoma could improve surgical decision-making.

Immunohistochemistry (IHC) generates a definitive diagnosis of CNS lymphoma using antibodies against lymphoma-specific proteins, such as CD20.¹² Unfortunately, IHC is a multistep staining procedure too slow for intraoperative feedback. Therefore, molecular probes that identify CNS lymphoma more efficiently than IHC may expedite diagnosis.³

Aptamers are a class of nanomolecules that bind molecular targets with antibody-like affinity .^{13,14} Unlike antibodies, aptamers are small and can readily diffuse through tissue samples to quickly bind their targets. Aptamers can be conjugated to fluorophores for molecular imaging and to chemotherapeutics for targeted therapy.^{3,15} Aptamers are possible reagents for histopathological tissue assessments; however, development of aptamer-based diagnostics remains in its infancy.

A lymphoma-specific, aptamer-based, conformational-switching molecular probe, TD05, that could identify human CNS lymphoma in animal brain tumor biopsies within 60 minutes was previously described.³. The binding location of this aptamer on B-cell lymphoma cells, its strong overlap with CD20 immunostaining, and its lack of binding to T cells have been previously reported by our group and others.^{3,16,17} Described herein is an optimize protocol using this truncated aptamer to generate a tissue-specific diagnosis of CNS lymphoma in 20 minutes or less from the time of biopsy.

Methods

TD05-488 Preparation

The DNA oligonucleotide, which had been isolated with high-performance liquid chromatography purification, was purchased from Integrated DNA Technologies Inc. A truncated version of the TD05 aptamer was used in our study.³ The sequence of the fluorophore-labeled aptamer is as follows: TD05-488: 5′/5Alex488N/AGGAGGATAGTTCGGTGGCTGTTCAGGGTCTCCTCCT-3′.^{17,18} (SEQ ID NO: 1) Aptamer probes were diluted to 3, 1, and 0.3 μM in aptamer binding buffer (6 mM MgCl₂, 1.2 mM CaCl₂, 4.5 g/L glucose, and 0.2% NaN₃ in 1× phosphate-buffered saline buffer) and annealed by heating at 94° C. for 5 minutes, followed by immediate chilling on ice for 10 minutes.

Fluorescent Tumor Preparation

Human glioma cells (U251) and human CNS lymphoma cells (Ramos) were acquired from American Type Culture Collection. U251 cells were incubated in DMEM supplemented with 10% fetal bovine serum (FBS) and Ramos cells in RPMI 1640 medium supplemented with 10% FBS at 37° C. in a humidified incubator with 5% carbon dioxide. U251 and Ramos cells were transduced with a lentivirus for stable expression of red fluorescent protein (RFP) under puromycin selection (Gentarget Inc.).

Animals

Nude mice (n=8) were obtained from The Jackson Laboratory and housed at the Barrow Neurological Institute's animal care facilities. All experiments were performed under the guidelines and regulations set forth by the National Institutes of Health Guide for the Care and Use of Laboratory Animals and approved by the Institutional Animal Care and Use Committee of the Barrow Neurological Institute at St. Joseph's Hospital and Medical Center.

Intracranial Implantation

Nude mice (6-7 weeks of age) were anesthetized with an intraperitoneal injection of ketamine (10 mg/kg) and xylazine (80 mg/kg), placed in a stereotactic apparatus (Kopf Instruments), and incised over the cranial midline. A burr hole was made 0.1 mm posterior to the bregma and 2.3 mm to the right of the midline. A needle was inserted to a depth of 3 mm and withdrawn 0.4 mm to a depth of 2.6 mm. Red fluorescent protein-expressing U251 or Ramos cells were infused over the course of 3 minutes. The burr hole was closed with bone wax, and the incision was sutured.

Acute Brain Slices

Rodents were deeply anesthetized with isoflurane 21-28 days post-tumor implantation and rapidly decapitated. Their brains were immediately removed and sectioned into 350-₁1m sections with a Leica VT1200 vibratome containing artificial cerebrospinal fluid (aCSF; in mM: 126 NaCl, 26 NaHCO₃, 2.5 KCl, 1.25 NaH₂PO₄, 2 MgSO₄, 2 CaCl₂, and 10 glucose; pH 7.4). Acute slices (n=28) were maintained at 37° C. in aCSF until aptamer staining.

Fluorescent Aptamer Staining Protocol

The annealed aptamers were mixed with yeast transfer RNA (tRNA; 0.1 mg/ml), which was used to block nonspecific binding. One milliliter of the prepared aptamer mixture was applied to submerse the acute tissue slices in a glass bottom dish and incubated on ice for 20, 10, or 5 minutes. Staining solution was then aspirated from the staining dish, and the tissue slices were quickly rinsed with 1 ml of ice-cold aptamer binding buffer for 1 minute before fluorescence imaging.

Fluorescence Imaging

Following aptamer incubation, dishes containing the acute slices were placed on the stage of a Zeiss 710 confocal laser scanning microscope. TD05-488 was imaged with 488 nm excitation and 505-525 nm emission. Red fluorescent protein was imaged with 560 nm excitation and 575-640 nm emission. Images were obtained with a Zeiss 20x/0.8 NA dry objective and confocal aperture of 1 Airy unit. The frame size was set to sample at the Nyquist rate. The laser and gain were set to fill the dynamic range of the photomultiplier tube in regions of strong fluorescence, and settings were maintained for all regions within each acute slice. For each slice, 7 regions of interest (ROIs) of 141×141 μm were imaged from the area of tumor implantation and 3 ROIs from contralateral normal brain.

Histology and IHC

Rodent xenograft brains containing CNS B-cell lymphoma or glioma were fixed in 4% paraformaldehyde and then embedded in paraffin. The brains were sectioned (8 lymphoma sections, 8 glioma sections) and stained with H & E. Additionally, the sections were counterstained for CD20 antibody (0.93 mg/L, Leica Biosystems Inc.) with a Bond III automated slide stainer (Lecia Biosystems Inc.). Sections were mounted on slides with optical glass and imaged with a 20× objective from an Olympus BX51 brightfield microscope (Olympus America).

Image Analysis

All image processing was completed utilizing linear functions in ImageJ (US National Institutes of Health).¹⁹ Regions of interest were randomly selected from areas of green fluorescence within each image. For each ROI, the number of cells expressing RFP and the number of cells with ring-like green fluorescence were quantified utilizing stereology approaches, as previously described.^{3,20} The percent of tumor cells labeled with the fluorescent aptamer was determined by quantifying the percent of RFP-expressing cells labeled with green fluorescence per ROI.

Clinician Image Evaluation

A file of 27 random ROIs was generated from images obtained from regions of CNS lymphoma, glioma, and normal brain incubated with 1-μM TD05-488 for 10 minutes. As a reference, an image of CNS lymphoma labeled with a fluorescent antibody was included. This file was distributed to neurosurgeons and pathologists, and their ability to distinguish CNS lymphoma from controls was quantified. The clinicians were blinded to the diagnosis of each image.

Statistical Analysis

Statistical analysis was performed with Prism version 7 for Windows (GraphPad Software). Data containing three or more groups were analyzed with ANOVA. A post hoc Tukey's multiple comparisons test was utilized to assess for significance in the difference between means. A Student t-test was implemented to identify differences when only two groups were compared. The alpha value was set for 0.05 for all tests. A logit model for data analysis was considered. However, percentages were maintained for simplicity and for a lack of extremes in the data set.

Results

Optimization of Staining Time and Aptamer Concentration

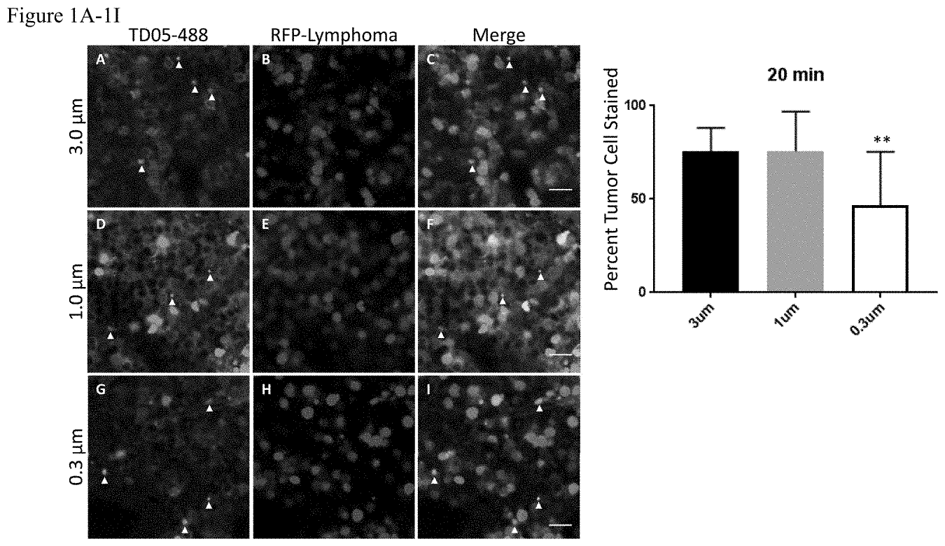

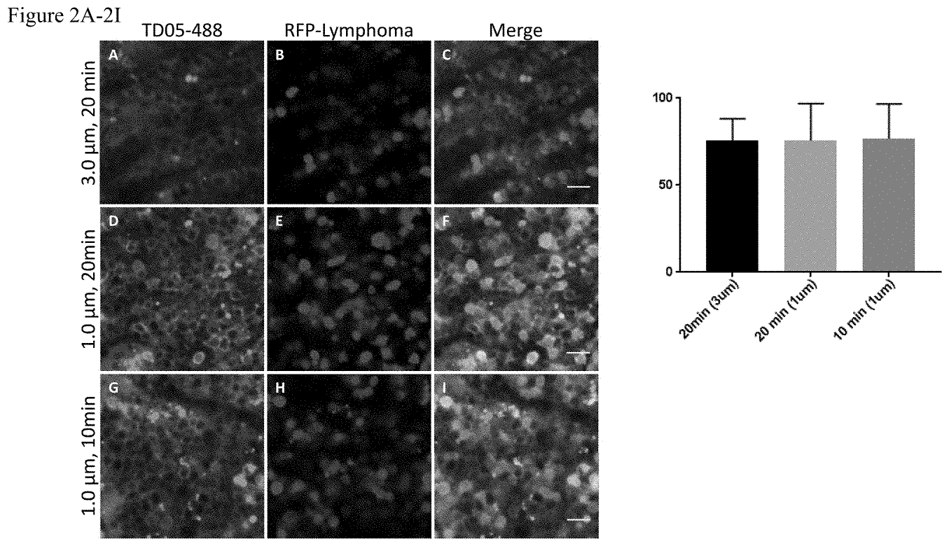

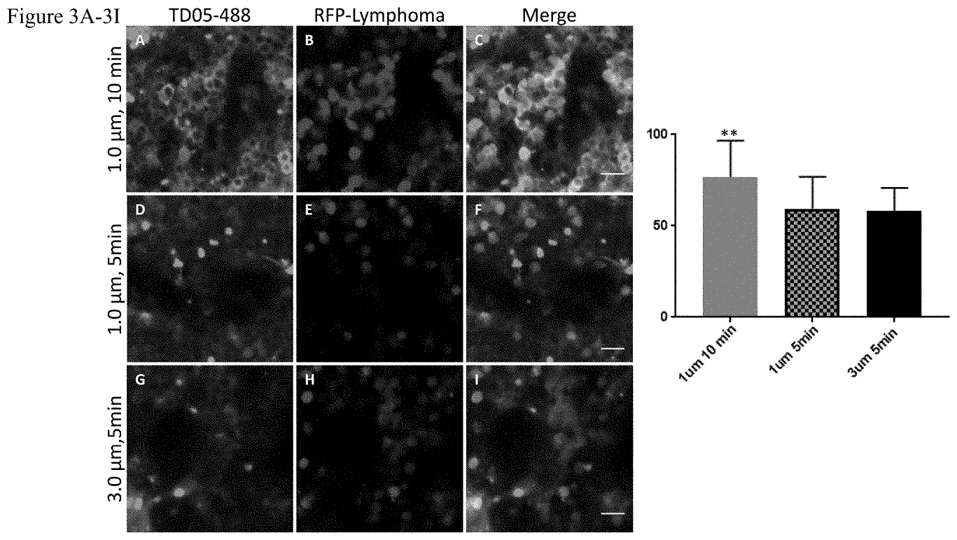

To develop an aptamer staining protocol that could identify CNS lymphoma quickly after frozen section, the staining efficacy of three concentrations of fluorescent aptamer at 20 minutes: 0.3, 1.0, and 3.0 μM was evaluated first. The 3.0- and 1.0-μM concentrations provided strong subjective staining of lymphoma cells and labeled 78% versus 77% of cells (p=0.99), respectively, at this time point. However, 0.3-μM TD05-488 labeled only 47% of tumor cells, which was significantly less than 3.0- and 1.0-μM TD05-488 at 20 minutes (p<0.001; FIG. 1). Staining efficacy of 1.0 μM at 10 minutes was tested and no significant difference was found compared to 1.0 and 3.0 μM at 20 minutes (p=0.97; FIG. 2). Given that 1.0 μM provided interpretable staining at 10 minutes, the staining efficacy of 3.0 and 1.0 μM at 5 minutes was evaluated next. Compared to 1.0 μM at 10 minutes, there was significantly less labeling with a 5-minute protocol utilizing these concentrations of TD05-488 (p<0.001; FIG. 3). Given these study findings, an aptamer concentration of 1.0 μM with a staining time of 10 minutes followed by a 1-minute wash was selected for testing with control samples; this protocol was most efficient, labeling 76.7% ±15.1% of lymphoma cells. For clarity, fluorescent artifacts are identified in FIG. 1. As previously reported, these artifacts are small areas of high fluorescent intensity that lack a structural ring-like staining pattern.³ Artifacts are present but not labeled in FIGS. 2 and 3.

Astrocytoma

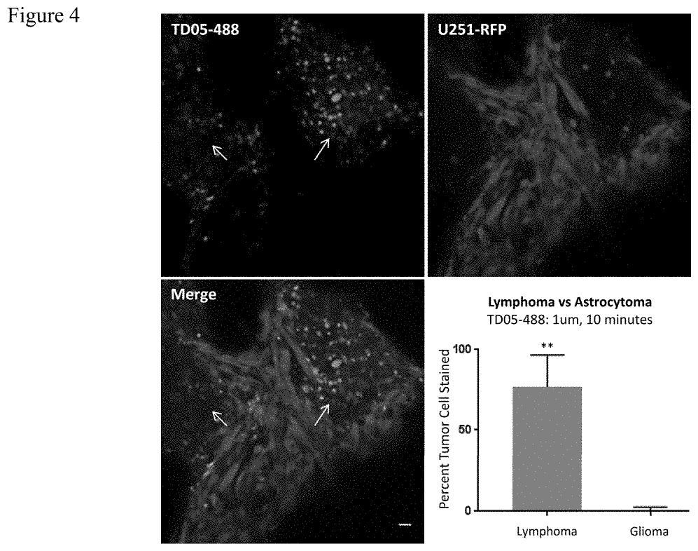

To evaluate nonspecific staining of our fluorescent aptamer on a negative control tumor, acute slices from rodents implanted with human U251 glioma cells expressing RFP were generated. Three acute slices from each of 3 rodents were incubated for 10 minutes with 1.0-μM TD05-488. Seven ROIs were imaged from each acute slice, and cellular fluorescence was quantified. FIG. 4 shows less than 1% fluorescence staining resembling CD20-positive lymphoma staining in these samples, showing a 10-minute staining protocol with 1.0-μM TD05-488 yielded a false-positive rate of 0.81% ±1.75% and labeled significantly fewer astrocytoma cells than lymphoma cells (p<0.001; FIG. 4).

Standard Histology

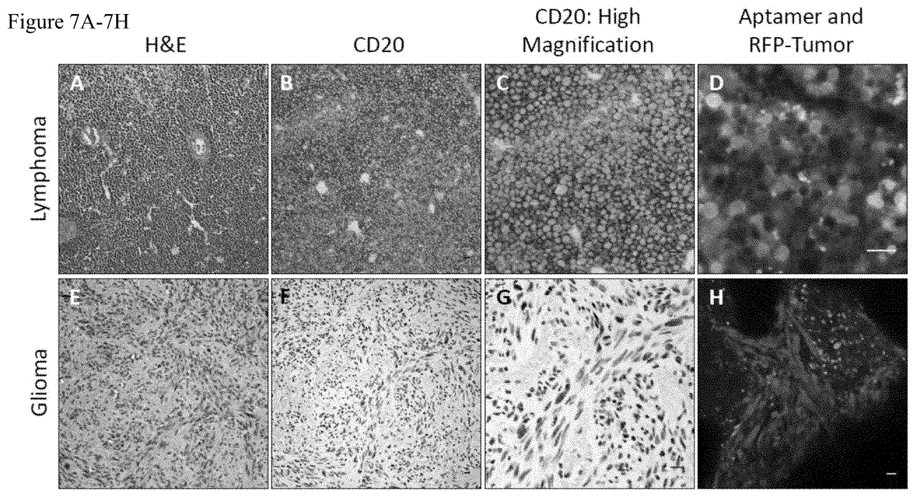

Brain sections from CNS B-cell lymphoma and glioma xenografts were processed for traditional H & E and CD20 antibody staining. The H & E staining showed regions of hypercellular tumor in lymphoma and glioma specimens (FIG. 7A and E). Lymphoma sections showed strong CD20 staining (FIG. 7B and C), whereas glioma samples lacked CD20 staining (FIG. 7F and G). High magnification revealed a ring-like staining pattern of CD20 in lymphoma samples (FIG. 7C) and a similar aptamer staining pattern of RFP-expressing B-cell lymphoma cells (FIG. 7D). Glioma samples lacked the ring-like staining pattern visualized from lymphoma cells (FIG. 7G and H).

Additional Controls: Normal Brain and Nonspecific Aptamer

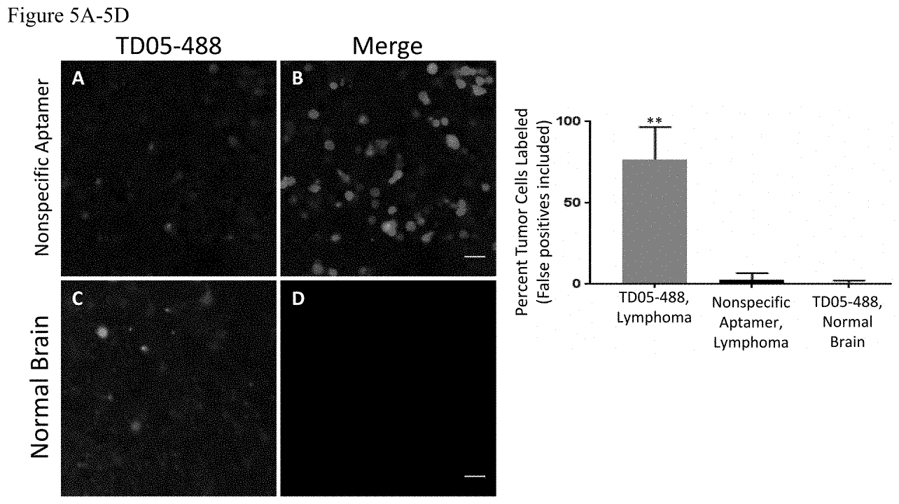

Additional controls, evaluated a 10-minute staining protocol with 1.0-μM TD05-488 on normal brain and assessed the staining of a nonspecific Alexa Fluor 488-conjugated aptamer on positive control biopsies. Our nonspecific fluorescent aptamer did not generate appreciable staining in lymphoma acute slices incubated in 1.0 μM for 10 minutes (2.71% ±3.72% cells labeled; FIGS. 5A and B). TD05-488 labeled 0.80% ±1.27% cells in normal brain (FIG. 5C and D).

Clinician Image Review

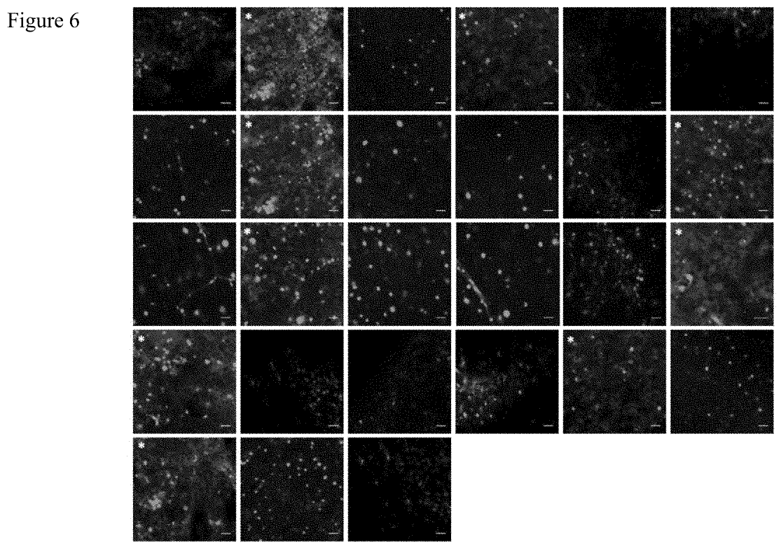

Two board-certified neurosurgeons (P.N. and S.Y.) and two fellowship-trained clinical pathologists (J. E. and Hany Osman) were evaluated in their interpretations of aptamer-based fluorescence images by distributing an image file containing 27 random samples incubated with our 10-minute TD05-488 staining protocol (FIG. 6). The images contained ROIs from lymphoma, astrocytoma, and normal brain. Clinicians were asked if each image contained CNS lymphoma or not. Overall, each clinician identified all lymphoma cases correctly. In this analysis, there were no false positives and no false negatives, and the intrarater and interrater correlations showed perfect agreement with a correlation of 1.0.

Discussion

Described herein is a truncated fluorescence aptamer providing specific diagnosis of xenograft CNS lymphoma within 11 minutes of biopsy (FIG. 2), a time frame that can provide meaningful intraoperative feedback. Our previous report indicated that a conformational-switching fluorescent aptamer could specifically diagnose CNS lymphoma from rodent xenograft biopsies within 45-60 minutes³—faster than IHC but too slow for effective intraoperative feedback.

We evaluated three concentrations of TD05-488 at three times points (5, 10, and 20 minutes, followed by a 1-minute wash) and found that a 1.0-μM staining protocol at 10 minutes labeled 77% of CNS cells with a false-positive rate under 1% from negative control astrocytoma biopsies (FIG. 4). Though staining of 80% or greater in positive controls was not observed, staining with 1.0-μM TD05-488 at 10 minutes provided consistent differentiation of CNS lymphoma from negative control samples for neurosurgeons and clinical pathologists.

Staining samples with TD05-488 was straightforward compared to the multiple steps required for frozen sections and IHC: tissue samples were placed in solution containing 1.0-μM TD05-488, rinsed for 1 minute, and then immediately imaged. Ex vivo imaging with the Zeiss 710 confocal microscope further improved time to diagnosis by eliminating the sectioning and slide preparation required for frozen sections and IHC. In comparison, an overnight staining protocol was required to complete standard IHC staining for samples viewed with brightfield imaging in this study (FIG. 7). Clinical ex vivo confocal imaging is a burgeoning field promising expedited histopathological diagnoses.^{21,22} The development of rapid molecular probes, such as TD05-488, should increase the clinical utility of this imaging modality.

There are barriers to implementing TD05-488 as a clinical diagnostic agent. Though ex vivo confocal microscopy increased the speed of interrogating samples after aptamer incubation, few pathology departments possess this imaging capability, and acquiring these microscopes can be expensive. Currently, fluorescence imaging techniques are showing utility in clinical pathology. As applications for fluorescence diagnostics increase, clinical fluorescence imaging devices capable of optical sectioning, such as confocal microscopes, may become more affordable and commonplace in pathology departments. Additionally, though TD05-488 provided reliable identification of CNS lymphoma from xenograft biopsies, a rigorous clinical trial is required to determine its efficacy on clinical specimens. Fluorescent artifacts were identified in a majority of our aptamer-labeled images (FIGS. 1 and 4). However, these artifacts did not resemble ring-like CD20 staining and did not influence diagnoses within our blinded image set (FIG. 6).

Conclusions

Intraoperative differentiation of CNS lymphoma from other malignant brain tumors, such as astrocytomas, remains a challenge in neuro-oncology. As a proof of concept shown here, ex vivo confocal imaging with the fluorescent aptamer TD05-488 can diagnose CNS lymphoma within 11 minutes of biopsy from xenograft brain tumor models. This procedure was straightforward and required fewer preparation steps than frozen sections or IHC. Clinical application of TD05-488, as well as other similar aptamers, may improve patient care by providing physicians with definitive intraoperative diagnoses.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### REFERENCES

1. Ziv E, Durack J C, Solomon S B. The importance of biopsy in the era of molecular medicine. Cancer J. 2016; 22(6):418-422.
2. Carnevale J, Rubenstein J L. The challenge of primary central nervous system lymphoma. Hematol Oncol Clin North Am. 2016; 30(6):1293-1316.
3. Georges J F, Liu X, Eschbacher J, et al. Use of a conformational switching aptamer for rapid and specific ex vivo identification of central nervous system lymphoma in a xenograft model. PLoS One. 2015; 10(4):e0123607.
4. Koriyama S, Nitta M, Shioyama T, et al. Intraoperative flow cytometry enables the differentiation of primary central nervous system lymphoma from glioblastoma. World Neurosurg. 2018; 112:e261-e268.
5. Scott B J, Douglas V C, Tihan T, et al. A systematic approach to the diagnosis of suspected central nervous system lymphoma. JAMA Neurol. 2013; 70(3):311-319.
6. Owen C M, Linskey M E. Frame-based stereotaxy in a frameless era: current capabilities, relative role, and the positive and negative predictive values of blood through the needle. J Neurooncol. 2009; 93(1): 139-149.

7. Davis M E. Glioblastoma: overview of disease and treatment. Clin J Oncol Nurs. 2016; 20(5)(suppl): S2-S8.
8. Fernandes C, Costa A, Osorio L, et al. Current standards of care in glioblastoma therapy. In: De Vleeschouwer S, ed. Glioblastoma. Codon; 2017.
9. Chand P, Amit S, Gupta R, Agarwal A. Errors, limitations, and pitfalls in the diagnosis of central and peripheral nervous system lesions in intraoperative cytology and frozen sections. J Cytol. 2016; 33(2):93-97.
10. Tofte K, Berger C, Torp S H, Solheim O. The diagnostic properties of frozen sections in suspected intracranial tumors: a study of 578 consecutive cases. Surg Neurol Int. 2014; 5:170. 11. Amraei R, Moradi A, Zham H, et al. A comparison between the diagnostic accuracy of frozen section and permanent section analyses in central nervous system. Asian Pac J Cancer Prey. 2017; 18(3):659-666.
12. Raoux D, Duband S, Forest F, et al. Primary central nervous system lymphoma: immunohistochemical profile and prognostic significance. Neuropathology. 2010; 30(3):232-240.
13. Ellington A D, Szostak J W. In vitro selection of RNA molecules that bind specific ligands. Nature. 1990; 346(6287):818-822.
14. Keefe A D, Pai S, Ellington A. Aptamers as therapeutics. Nat Rev Drug Discov. 2010; 9(7):537-550.
15. Hicke B J, Stephens A W, Gould T, et al. Tumor targeting by an aptamer. J Nucl Med. 2006; 47(4): 668-678.
16. Mallikaratchy P, Tang Z, Kwame S, et al. Aptamer directly evolved from live cells recognizes membrane bound immunoglobin heavy mu chain in Burkitt's lymphoma cells. Mol Cell Proteomics. 2007; 6(12):2230-2238.
17. Mallikaratchy P R, Ruggiero A, Gardner J R, et al. A multivalent DNA aptamer specific for the B-cell receptor on human lymphoma and leukemia. Nucleic Acids Res. 2011; 39(6): 2458-2469.
18. Tang Z, Mallikaratchy P, Yang R, et al. Aptamer switch probe based on intramolecular displacement. J Am Chem Soc. 2008; 130(34):11268-11269.
19. Schneider C A, Rasband W S, Eliceiri K W. NIH Image to ImageJ: 25 years of image analysis. Nat Methods. 2012; 9(7): 671-675.
20. Georges J F, Martirosyan N L, Eschbacher J, et al. Sulforhodamine 101 selectively labels human astrocytoma cells in an animal model of glioblastoma. J Clin Neurosci. 2014; 21(5): 846-851.
21. Martirosyan N L, Georges J, Eschbacher J M, et al. Confocal scanning microscopy provides rapid, detailed intraoperative histological assessment of brain neoplasms: experience with 106 cases. Clin Neurol Neurosurg. 2018; 169:21-28.
22. Mooney M A, Georges J, Yazdanabadi M I, et al. Immediate ex-vivo diagnosis of pituitary adenomas using confocal reflectance microscopy: a proof-of-principle study. J Neurosurg. 2018; 128(4): 1072-1075.

1. A method of detecting the presence of B-cell lymphoma in a tumor sample from a subject, the method comprising:
contacting the tumor sample with an aptamer comprising at least 90% sequence identity to SEQ ID NO. 1, wherein the aptamer comprises a detectable label, to create a treated tumor sample;
washing the treated tumor sample; and
imaging the treated tumor sample, wherein the presence of the labeled aptamer is indicative of B-cell lymphoma.

2. The method of claim 1, wherein the tumor is one or more of a brain tumor, a spinal cord tumor, an eye tumor, a meningeal tumor, or a leptomeningeal tumor.

3. The method of claim 1, wherein tumor sample comprises a biopsy section.

4. The method of claim 1, wherein the aptamer has a concentration of between 0.3 μM and 3 μM, inclusive.

5. The method of claim 1, wherein the tumor sample is contacted with the aptamer for between 5 and 20 minutes, inclusive.

6. The method of claim 1, wherein the tumor sample is contacted with an agent to block nonspecific binding in addition to the aptamer.

7. The method of claim 6, wherein the agent to block nonspecific binding is yeast transfer RNA (tRNA).

8. The method of claim 1, wherein the label is a fluorescent label, luminescent label, radioisotopic label, or a label that comprises an enzyme capable of a colorimetric conversion of a substrate.

9. The method of claim 1, wherein the subject has a brain tumor, the tumor sample comprises a fresh biopsy section, the aptamer is SEQ ID NO: 1 and comprises a fluorescent label, and the method, including imaging, is completed in 20 minutes or less.

10. A method of treating B-cell lymphoma in a subject in need thereof, the method comprising:
contacting a tumor sample from the subject with an aptamer with at least 90% sequence identity to SEQ ID NO. 1, wherein the aptamer comprises a detectable label, to create treated sample, washing the treated tumor sample, imaging the treated tumor sample, and treating the patient based on the presence or absence of aptamer in the treated tumor sample.

11. The method of claim 10, wherein imaging the treated tumor sample reveals the presence of aptamer staining, wherein treating the subject comprises a surgical or non-surgical medical intervention.

12. The method of claim 10, wherein the aptamer has a concentration of between 0.3 μM and inclusive.

13. The method of claim 10, wherein the tumor sample comprises a biopsy section and the biopsy sections are contacted with the aptamer for between 5 and 20 minutes, inclusive and contacted with yeast transfer RNA (tRNA) in addition to the aptamer.

14. The method of claim 10, wherein the tumor comprises a brain tumor.

15. A method of preparing a tumor sample for imaging, the method comprising:
contacting the tumor sample with an aptamer comprising at least 90% sequence identity to SEQ ID NO. 1, wherein the aptamer comprises a detectable label, to create a treated tumor sample; and
washing the treated tumor sample.

16. The method of claim 15, wherein the tumor comprises a brain tumor.

17. The method of claim 15, wherein tumor sample comprises a biopsy section.

18. The method of claim 15, wherein the aptamer has a concentration of between 0.3 mM and 3 mM, inclusive.

19. The method of claim 15, wherein the aptamer has a concentration of between 0.3 μM and 1 μM, inclusive.

20. The method claim 15, wherein the tumor sample contacted with the aptamer for between 5 and 10 minutes, inclusive.


SEQUENCE LISTING (1 sequence)

SEQ ID NO: 1 - DNA, 37 bp, synthetic construct
  source 1..37: mol_type = other DNA
aggaggatagttcggtggctgttcagggtctcctcct